(19) United States
(12) Patent Application Publication
(10) Pub. No.: US 2005/0106190 A1
(43) Pub. Date: May 19, 2005

(54) Cosmetic and/or pharmaceutical emulsions

(76) Inventors: Rolf Kawa, Monheim (DE); Lars Zander, Duesseldorf (DE)

Correspondence Address: COGNIS CORPORATION;PATENT DEPARTMENT, 300 BROOKSIDE AVENUE, AMBLER, PA, 19002, US

(21) Appl. No.: 10/498,796

(22) PCT Filed: Dec. 10, 2002
(86) PCT No.: PCT/EP02/13984

(30) Foreign Application Priority Data
Dec. 18, 2001 (DE) 101 62 351.8

(51) Int. Cl. A61K 7/075; A61K 7/08

(52) U.S. Cl. 424/401; 424/70.31

(57) ABSTRACT

Polyalkylene ethers comprised of C₅₋₁₀₀ alkylene groups and having an average molecular weight of from 300 to 100000, which can be prepared by polycondensation of hydroxy-functional reactants selected from the group consisting of diols and polyols are described in addition to their use in cosmetic and/or pharmaceutical preparations.

### FIELD OF THE INVENTION

This invention relates to cosmetic and/or pharmaceutical emulsions containing special polyalkylene ethers and to the use of these polyalkylene ethers in cosmetic/pharmaceutical preparations in general and in sun protection preparations in particular for improving the water resistance of these preparations.

### PRIOR ART

Although emulsions have been known for some time, intensive efforts are constantly being made on the cosmetic market to improve both the stability and the sensory properties of these disperse systems. Present trends include inter alia the search for new oil components and polymers which may readily be incorporated in emulsions, which allow the formulation of particularly storage-stable emulsions and which, in sensory terms, leave the skin feeling lighter.

The water resistance of the preparations is another key factor for special applications, for example sun protection products, because the UV filters are intended to remain on the skin for as long as possible without being washed off during bathing. The water resistance of a sun protection formulation is normally achieved by the addition of polymers, for example PVP/Hexadecene Copolymer (Antaron® V-216). Unfortunately, these polymers have the disadvantage that the water resistance they provide is short-lived and long-term resistance, as required for example in watersports (surfing) and in sun protection for children, cannot be achieved. In addition, the sensory properties of the emulsion in terms of absorption, spreadability and tackiness are seriously affected.

Accordingly, the problem addressed by the present invention was to provide emulsions based on new polymers which would provide the emulsions with improved sensory properties, more particularly in regard to absorption, spreadability and tackiness. Another problem addressed by the invention was to develop formulations which, compared with the prior art, would show improved water resistance, i.e. would afford improved long-term protection where UV filters are present in the formulations.

### DESCRIPTION OF THE INVENTION

It has now surprisingly been found that cosmetic preparations based on special polyalkylene ethers have improved sensory behavior in regard to spreadability, absorption and tackiness. The polyalkylene ethers can readily be incorporated in emulsions and provide the preparations according to the invention with improved water resistance.

Accordingly, the present invention relates to cosmetic and/or pharmaceutical preparations containing polyalkylene ethers with C₅₋₁₀₀ alkylene groups and an average molecular weight of 300 to 100,000 and preferably in the range from 500 to 50,000. The molecular weights can be determined by gel permeation chromatography. The present invention also relates to the use of these polyalkylene ethers in cosmetic and/or pharmaceutical preparations, more particularly for improving the water resistance of the resulting preparations. This is of major interest for sun protection formulations in particular, but is also relevant to many other types of decorative cosmetics, such as for example mascara, eye shadow, waterproof make-up, eyeliner, kajal sticks, etc.

### POLYALKYLENE ETHERS

Polyalkylene ethers may be represented by the following general structural formula: 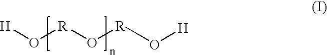
Polyalkylene ethers belong to the polyethers which are a large group of structurally very different polymers that includes the polyalkylene glycols (polyethylene glycols, polypropylene glycols and polyepichlorohydrins), epoxy resins, polytetrahydrofurans (polytetramethylene glycols), polyoxetanes, polyphenylene ethers or polyether ketones. Polyalkylene ethers or polyalkylene glycols are understood to be predominantly linear polyethers, i.e. polymers with terminal hydroxy groups. Technically important representatives of these polyether polyols are the polyethylene glycols, polypropylene glycols and polytetramethylene glycols (polytetrahydrofurans) which are produced by polyaddition of ethylene oxide, propylene oxide or tetrahydrofuran onto water. Block copolymers of ethylene and propylene oxide have acquired relatively major technical significance (Pluronic®).

The polyalkylene ethers with C₅₋₁₀₀ lalkylene groups suitable for use in accordance with the invention may be prepared by polycondensation reactions from diols or polyols in the presence of sulfosuccinic acid as catalyst.

The diol component may be selected, in particular, from saturated or unsaturated, branched or unbranched, aliphatic dihydroxy compounds containing 5 to 36 carbon atoms or aromatic dihydroxy compounds, such as, for example, pentane-1,5-diol, hexane-1,6-diol, neopentyl glycol, bis-(hydroxymethyl)-cyclohexanes, bisphenol A, dimer diols, hydrogenated dimer diols or even mixtures of the diols mentioned. In addition, polyhydric alcohols may also be used in the polymerization reaction, including for example glycerol, di- and polyglycerol, trimethylolpropane, pentaerythritol or sorbitol.

Polyalkylene ethers with an average molecular weight of 300 to 100,000 are suitable for the purposes of the invention. Polyalkylene ethers with an average molecular weight of 500 to 50,000, more particularly 500 to 20,000, are particularly suitable, polyalkylene ethers with an average molecular weight of 1,000 to 5,000 being particularly preferred. Although polyalkylene ethers such as these are normally solid in consistency or viscous to tacky materials, they are easy to incorporate and, besides sensory advantages, such as reduced tackiness, provide the preparations according to the invention with improved water resistance (vide infra).

The preparations according to the invention preferably contain polyalkylene ethers obtained by polycondensation of a dimer diol or αω,-C₅₋₃₆ alkanediol. The polycondensation is preferably carried out in the presence of sulfosuccinic acid, but may also be carried out with other catalysts having the same effect. Dimer diols are mixtures from their production. Their production is well-known from the prior art, for example from DE 1 768 313 and U.S. Pat. No. 2,347,562. Preferred dimer diol components for the reaction leading to the polyalkylene ethers usable in accordance with the invention are dimer diols containing a total of 12 to 100 carbon atoms. C₁₂₋₄₀ dimer diols are particularly suitable, C₁₂₋₂₄ dimer diols are preferred and C₁₆₋₂₂ dimer diols are particularly preferred, the chain lengths mentioned here relating to one chain. Preparations based on polyalkylene ethers obtained by polycondensation of hydrogenated dimer diols with iodine values of 20 to 80 and preferably 50 to 70 are preferred for the purposes of the invention. According to the invention, the use of Pripol® 2033 (Uniqema) and Speziol® 36/2 (Cognis Deutschland GmbH) is particularly preferred for the polycondensation leading to the polyalkylene ethers.

C₂₋₁₈ diols, for example decane-1,10-diol and dodecane-1,12-diol, are preferably used as the α,ω-alkanediol component. According to the invention, alkane-C₂₋₁₂-diols, more particularly alkane-C₆₋₁₂-diols, are suitable for the purposes of the invention. Mixtures of α,ω-hexanediol and α,ω-dodecanediol are particularly preferred as the α,ω-alkanediol component for the polycondensation.

The polyalkylene ethers are normally used in quantities of 0.1 to 20% by weight, preferably in quantities of 1 to 10% by weight and more particularly in quantities of 1 to 5% by weight, based on the final formulation of the cosmetic preparation.

Cosmetic and/or Pharmaceutical Preparations

The preparations according to the invention may be formulated as substantially water-free oils, creams, gels, lotions, alcoholic and aqueous/alcoholic solutions, emulsions, sprayable emulsions, wax/fat compounds, stick preparations and the like. Accordingly, the compositions according to the invention have viscosities varying from 100 to 1,000,000 mPa.s (Brookfield RVF, 23° C., spindle and r.p.m. dependent on viscosity according to the manufacturer). The preparations according to the invention preferably have a viscosity of 100 to 300,000 mPa.s at 23° C. Depending on the particular application, the preparations according to the invention contain a number of other auxiliaries and additives, such as for example oil components, emulsifiers, surfactants, pearlizing waxes, consistency factors, thickeners, superfatting agents, stabilizers, polymers, silicone compounds, fats, waxes, lecithins, phospholipids, biogenic agents, UV protection factors, antioxidants, deodorizers, antiperspirants, anti-dandruff agents, film formers, swelling agents, insect repellents, self-tanning agents, tyrosine inhibitors (depigmenting agents), hydrotropes, solubilizers, preservatives, perfume oils, dyes and the like.

Oil Components

The preparations according to the invention preferably also contain at least one oil component. Oil components in the context of the invention are substances which are liquid at 20° C. and immiscible with water at 25° C. or mixtures of such substances. The combination with oil components enables the sensory properties of the preparations to be optimized. The quantity of oil components in the composition as a whole can vary between 1 and 98% by weight according to the particular formulation (for example oil, cream, lotion, sprayable emulsion). In a preferred embodiment, the preparations according to the invention contain 1 to 30% by weight oil components and more particularly 5 to 30% by weight oil components.

Suitable oil components are, for example, Guerbet alcohols based on fatty alcohols containing 6 to 18 and preferably 8 to 10 carbon atoms (for example Eutanol® G), esters of linear C₆₋₂₂ fatty acids with linear or branched C₆₋₂₂ fatty alcohols, esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₃₋₃₈ alkylhydroxycarboxylic acids with linear or branched C₆₋₂₂ fatty alcohols, more especially diethylhexyl malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols Hydagen® HSP, Sovermol® 750, Sovermol® 1102), silicone oils (cyclo-methicone, silicon methicone types, etc.) and/or aliphatic or naphthenic hydrocarbons, for example mineral oil, vaseline, petrolatum, squalane, squalene or dialkyl cyclohexanes.

A distinct improvement in the sensory properties of the compositions according to the invention is achieved when dialkyl ethers and/or dialkyl carbonates are used as oil components either on their own or in conjunction with other oil components. According to the invention, therefore, dialkyl ethers and/or dialkyl carbonates are preferred oil components. In addition, silicone compounds may advantageously be used to prevent the unwanted so-called “white residues” (microfoam formation) in cosmetic formulations. For example, cyclomethicone and dimethicone are used in quantities of 1 to 20% by weight, based on the composition as a whole, for this purpose.

Surfactants/Emulsifiers

In another preferred embodiment, the preparations according to the invention additionally contain at least one emulsifier. The addition of emulsifiers improves the incorporation of the polyalkylene ethers.

In a preferred embodiment, the preparations according to the invention contain at least one nonionic emulsifier. Nonionic emulsifiers are distinguished by their kindness to the skin, their mildness and their ecotoxicologically favorable properties. In addition, the stability and sensory properties of the compositions according to the invention can be improved by the use of a combination of nonionic w/o and o/w emulsifiers. A particularly preferred combination is commercially available as Eumulgin® VL 75 (Cognis Deutschland GmbH). The compositions according to the invention contain the emulsifier(s) in a quantity of typically 0.1 to 15% by weight, preferably 1 to 10% by weight and more particularly 3 to 10% by weight, based on the total weight of the composition.

Nonionic Emulsifiers

The group of nonionic emulsifiers include:
(1) products of the addition of 2 to 50 mol ethylene oxide and/or 0 to 20 mol propylene oxide onto linear C₈₋₄₀ fatty alcohols, onto C₁₂₋₄₀ fatty acids and onto alkyl phenols containing 8 to 15 carbon atoms in the alkyl group;
(2) C_{12/18} fatty acid mono- and diesters of addition products of 1 to 50 mol ethylene oxide into glycerol;
(3) glycerol mono- and dieters and sorbitan mono- and diesters of saturated and unsaturated C₆₋₂₂ fatty acids and ethylene oxide addition products thereof;
(4) alkyl mono- and oligoglycosides containing 8 to 22 carbon atoms in the alkyl group and ethoxylated analogs thereof;
(5) addition products of 7 to 60 mol ethylene oxide onto castor oil and/or hydrogenated castor oil;
(6) polyol esters and, in particular, polyglycerol esters such as, for example, polypolypoly-1 2-hydroxystearate, polyglycerol polyricinoleate, polyglycerol diisostearate or polyglycerol dimerate. Mixtures of compounds from several of these classes are also suitable;
(7) products of the addition of 2 to 15 mol ethylene oxide onto castor oil and/or hydrogenated castor oil;
(8) partial esters based on linear, branched, unsaturated or saturated C₆₋₂₂ fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose) or mixed esters such as, for example, glyceryl stearate citrate and glyceryl stearate lactate;
(9) wool wax alcohols;
(10) polysiloxane/polyalkyl polyether copolymers and corresponding derivatives;
(11) mixed esters of pentaerythritol, fatty acids, citric acid and fatty alcohol and/or mixed esters of fatty acids containing 6 to 22 carbon atoms, methyl glucose and polyols, preferably glycerol, and
(12) polyalkylene glycols.

The addition products of ethylene oxide and/or propylene oxide with fatty alcohols, fatty acids, alkylphenols, glycerol monoesters and diesters and sorbitan monoesters and diesters of fatty acids or with castor oil are known, commercially available products. They are homolog mixtures of which the average degree of alkoxylation corresponds to the ratio between the quantities of ethylene oxide and/or propylene oxide and substrate with which the addition reaction is carried out. The products in question are w/o or o/w emulsifiers, depending on the degree of ethoxylation. Reaction products with 1 to 100 mol ethylene oxide are particularly suitable for the preparations according to the invention.

Polyolpoly-12-hydroxystearates and the mixtures thereof which are marketed under the names of “Dehymuls® PGPH” (w/o emulsifier) or “Eumulgin® VL 75” (mixture with Coco Glucosides in a ratio by weight of 1:1, o/w emulsifier) or Dehymuls® SBL (w/o emulsifier) by Cognis Deutschland GmbH) are also preferred for the purposes of the invention by virtue of their mildness. The polyol component of these emulsifiers may be derived from substances which contain at least 2, preferably 3 to 12 and more particularly 3 to 8 hydroxyl groups and 2 to 12 carbon atoms.

Suitable lipophilic w/o emulsifiers are, in principle, emulsifiers with an HLB value of 1 to 8 which are summarized in numerous tables and which are known to the expert. Some of these emulsifiers are listed, for example, in Kirk-Othmer, Encyclopedia of Chemical Technology, 3rd Edition, 1979, Vol. 8, page 913. For ethoxylated products, the HLB value can be calculated using the following equation: HLB=(100-L): 5, where L is the percentage by weight of the lipophilic groups, i.e. the fatty alkyl or fatty acyl groups, in % by weight in the ethylene oxide adducts.

Particularly advantageous w/o emulsifiers are partial esters of polyols, more particularly C₃₋₆ polyols such as, for example, glyceryl monoesters, partial esters of pentaerythritol or sugar esters, for example sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesqui-hydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable emulsifiers.

In cases where water-soluble active components and water are incorporated, at least one emulsifier from the group of nonionic o/w emulsifiers (HLB value: 8-18) and/or solubilizers should be used. Such emulsifiers and/or solubilizers are, for example, the ethylene oxide adducts mentioned at the beginning with a correspondingly high degree of ethoxylation, for example 10-20 ethylene oxide units for o/w emulsifiers and 20 to 40 ethylene oxide units for so-called solubilizers. According to the invention, Ceteareth-20 and PEG-20 Glyceryl Stearate are particularly advantageous o/w emulsifiers.

Nonionic emulsifiers from the group of alkyl oligoglycosides are particularly kind to the skin and, accordingly, are particularly suitable as o/w emulsifiers for the purposes of the invention. They enable the sensory properties of the compositions to be optimized and provide for particularly easy incorporation of the polyalkylene ethers. C₈₋₂₂ alkyl mono- and oligoglycosides, their production and their use are known from the prior art. They are produced in particular by reacting glucose or oligosaccharides with primary alcohols containing 8 to 22 carbon atoms, preferably 12 to 22 and more particularly 12 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based. Products available under the name of Plantacare® contain a C₈₋₁₆ alkyl group attached by a glucosidic bond to an oligoglucoside unit with an average degree of oligomerization of 1 to 2. The acyl glucamides derived from glucamine are also suitable nonionic emulsifiers. The product marketed under the name of Emulgade® PL 68/50 by Cognis Deutschland GmbH, which is a 1:1 mixture of alkyl polyglucosides and fatty alcohols, is preferred for the purposes of the invention. The mixture of Lauryl Glucoside, Polyglyceryl-2-Dipolyhydroxystearate, glycerol and water which is marketed under the name of Eumulgin® VL 75 may also be used with advantage for the purposes of the invention.

A particularly preferred embodiment of the composition according to the invention contains:
(a) 1 to 10% by weight polyalkylene ethers with C₅₋₁₀₀ alkylene groups and an average molecular weight of 300 to 100,000,
(b) 5 to 30% by weight oil components,
(c) 0.1 to 10% by weight emulsifier(s),
(d) 0 to 90% by weight water.

According to the invention, particularly preferred cosmetic preparations contain (a) 1 to 10% by weight polyalkylene ethers with an average molecular weight of 500 to 50,000 based on dimer diols, more particularly C₁₂₋₄₀ dimer diols, (b) 5 to 30% by weight oil components, (c) 0.1 to 10% by weight emulsifier(s) and 0 to 90% by weight water.

Other Surfactants/Emulsifiers

The compositions may also contain zwitterionic, amphoteric, cationic and anionic surfactants according to the application envisaged. The combination with selected anionic surfactants is particularly suitable for the purposes of the invention (vide infra).

Zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example coco-acylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is a particularly preferred zwitterionic surfactant.

Ampholytic surfactants are also suitable, particularly as co-surfactants. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylamino-butyric acids, N-alkyliminodipropionic acids, N-hydroxyethyl-N-alkyl-amidopropyl glycines, N-alkyl taurines, N-alkyl sarcosines, 2-alkyl-aminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

In another preferred embodiment, the preparations according to the invention contain at least one emulsifier selected from the group of anionic emulsifiers, more particularly alkyl phosphates and alkyl sulfates. Anionic emulsifiers/surfactants contribute towards further improving the water resistance of the preparations according to the invention.

Anionic surfactants are characterized by a water-solubilizing anionic group such as, for example, a carboxylate, sulfate, sulfonate or phosphate group and a lipophilic group. Dermatologically safe anionic surfactants are known to the expert in large numbers from relevant textbooks and are commercially available. They are, in particular, alkyl sulfates in the form of their alkali metal, ammonium or alkanolammonium salts, alkylether sulfates, alkylether carboxylates, acylisethionates, acyl sarcosinates, acyl taurines containing linear C₁₂₋₁₈ alkyl or acyl groups and sulfosuccinates and acyl glutamates in the form of their alkali metal or ammonium salts. Among the anionic surfactants, alkali metal salts of fatty acids (sodium stearate) and, in particular, alkyl sulfates (Lanefte® E) and alkyl phosphates (Amphisol® K) are particularly suitable for the preparations according to the invention because they lead to particularly stable and homogeneous emulsions with relatively high viscosities and contribute to a significant increase in water resistance.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as for example the dialkyl ammonium methosulfates and methyl hydroxyalkyl dialkoyloxyalkyl ammonium methosulfates marketed under the name of Stepantex® and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolyzates.

Humectants/Skin Moisturizers

In another preferred embodiment, the composition according to the invention also contains a humectant which contributes towards optimizing the sensory properties of the composition and which serves to regulate the skin moisture level. At the same time, the low-temperature stability of the preparations according to the invention, particularly in the case of emulsions, is increased. The humectants are normally present in a quantity of 0.1 to 15% by weight, preferably 1 to 10% by weight and more particularly 5 to 10% by weight.

According to the invention, suitable humectants are inter alia amino acids, pyrrolidone carboxylic acid, lactic acid and salts thereof, lactitol, urea and urea derivatives, uric acid, glucosamine, creatinine, cleavage products of collagen, chitosan or chitosan salts/derivatives and, in particular, polyols and polyol derivatives (for example glycerol, diglycerol, triglycerol, ethylene glycol, propylene glycol, butylene glycol, erythritol, 1,2,6-hexanetriol, polyethylene glycols, such as PEG4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolyzates and mixtures of hydrogenated wheat protein and PEG-20-acetate copolymer. According to the invention, particularly preferred humectants are glycerol, diglycerol and triglycerol.

UV Protection Factors and Antioxidants

A preferred embodiment of the preparations according to the invention relates to sun protection formulations, i.e. the preparations according to the invention additionally contain a UV filter. It has surprisingly been found that the C₅₋₁₀₀ polyalkylene ethers improve the water resistance of sun protection formulations and thus provide long-time protection in water.

Since ca. 60% UV-B and ca. 80% UV-A radiation (based on the UV reaching the earth's surface) is still active in water to a depth of 50 cm, the water resistance of sun protection emulsions is particularly important, especially for children and aquatic athletes. Effective sun protection emulsions should be made water-resistant, should stay firmly on the skin and should only be slowly washed off in water. According to the COLIPA recommendations, a sun protection formulation is water-resistant if at least 50% of the original sun protection effect is still present after exposure to water under defined conditions. The sun protection effect is achieved by the use of suitable UV filters.

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor as described in EP 0693471 B1;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzo-phenone, 2-hydroxy4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives as described in EP 0694521 B1.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol®) 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminum and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or; nanopigments are preferably used in sun protection products. Micronized zinc oxide is preferably used. Other suitable UV filters can be found in P. Finkel's review in SOFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Deodorant and Antiperspirant Components

Another preferred embodiment of the composition according to the invention additionally contains a deodorizing/antiperspirant component or a combination of such components. The water resistance of the preparations is also of importance in this embodiment, ensuring that the active components are not washed off by perspiration and do not adhere to clothing.

Such active components include astringent metal salts (antiperspirant components), germ inhibitors, enzyme inhibitors, odor absorbers, odor maskers or combinations of these active components. The deodorant/antiperspirant components are present in the compositions according to the invention in a quantity of 0.1 to 30% by weight, preferably in a quantity of 5 to 25% by weight and more particularly in a quantity of 10 to 25% by weight (based on the quantity of active substance).

Suitable antiperspirant components are, for example, aluminum chlorhydrates, aluminum/zirconium chlorhydrates and zinc salts. These antiperspirants probably act by partially blocking the sweat glands through the precipitation of proteins and/or polysaccharides. Besides the chlorhydrates, aluminum hydroxylactates and acidic aluminum/zirconium salts may also be used. For example, an aluminum chlorhydrate which corresponds to the formula [Al₂(OH)₅Cl] 2.5H₂O and which is particularly preferred for the purposes of the invention is commercially available under the name of Locron® from Clariant GmbH. The aluminum/zirconium tetrachlorohydrex/glycine complexes marketed, for example, by Reheis under the name of Rezal® 36G are also preferably used in accordance with the invention.

Other suitable deodorizers are esterase inhibitors, preferably trialkyl citrates, such as trimethyl citrate, tripropyl citrate, triisopropyl citrate, tributyl citrate and, in particular, triethyl citrate (Hydagen® CAT, Cognis Deutschland GmbH). Esterase inhibitors inhibit enzyme activity and thus reduce odor formation. The free acid is probably released through the cleavage of the citric acid ester, reducing the pH value of the skin to such an extent that the enzymes are inhibited. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester. Antibacterial agents which influence the germ flora and destroy or inhibit the growth of perspiration-decomposing bacteria, may also be present in the compositions. Examples of such antibacterial agents are chitosan, phenoxyethanol and chlorhexidine gluconate. 5-Chloro-2-(2,4-dichlorophenoxy)-phenol, which is marketed under the name of Irgasan® by Ciba-Geigy of Basel, Switzerland, has also proved to be particularly effective.

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo4-chlorophenol), 3-methyl-4-(1 -methylethyl)-phenol, 2-benzyl4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component.

Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, P-damascone, geranium oil bourbon, cyclohexyl salicylate, Vertofix Coeur, lso-E-Super, Fixolide NP, evernyl, iraldein gamma, phenylacetic acid, geranyl acetate, benzyl acetate, rose oxide, romillat, irotyl and floramat.

Insect Repellents

Another preferred embodiment of the composition according to the invention contains an insect repellent or a combination of insect repellents. The water resistance of the preparations is also important for this embodiment, ensuring that the active components are not washed off and long-term protection is developed.

Suitable insect repellents are N,N-diethyl-m-toluamide, pentane-1,2-diol, 3-(N-n-butyl-N-acetylamino)-propionic acid ethyl ester), which is marketed as Insect Repellent 3535 by Merck KGaA, and Ethyl Butylacetyl-aminopropionate. They are normally used in the compositions according to the invention in a quantity of 0.1 to 10% by weight, preferably in a quantity of 1 to 8% by weight and more particularly in a quantity of 2 to 6% by weight, based on the overall composition.

Viscosity Adjusters

The required viscosity for the compositions according to the invention is achieved by addition of viscosity adjusters. Viscosity adjusters additionally increase the water resistance of the preparations according to the invention. Accordingly, a preferred embodiment of the preparation according to the invention additionally contains at least one viscosity adjuster. Suitable viscosity adjusters are inter alia consistency factors such as, for example, fatty alcohols or hydroxyfatty alcohols containing 12 to 22 and preferably 16 to 18 carbon atoms and also partial glycerides, fatty acids containing 12 to 22 carbon atoms or 12-hydroxyfatty acids. A combination of these substances with alkyl oligoglucosides and/or fatty acid N-methyl glucamides of the same chain length and/or polyglycerol poly-12-hydroxystearates is preferably used because combinations such as these provide particularly stable and homogeneous emulsions. Other suitable viscosity adjusters are thickeners such as, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), uncrosslinked and polyol-crosslinked polyacrylic acids, polyacrylamides, polyvinyl alcohol and polyvinyl pyrrolidone. Other viscosity adjusters which have proved to be particularly effective are bentonites, for example Bentone® Gel VS-5PC (Rheox) which is a mixture of cyclopentasiloxane, Disteardimonium Hectorite and propylene carbonate. Other suitable viscosity adjusters are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable viscosity adjusters also include anionic, zwitterionic, amphoteric and nonionic copolymers such as, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, acrylamidopropyl trimethylammonium chloride/acrylate copolymers, octylacrylamide/methyl methacrylate/tert.-butylaminoethyl methacrylate/2-hydroxypropyl methacrylate copolymers, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil., 108, 95 (1993).

According to the invention, it is preferred to use polymers in quantities of 0.1 to 5% by weight, preferably in quantities of 0.1 to 3% by weight and more particularly in quantities of 0.1 to 2% by weight, based on the composition as a whole. Polyacrylic acid homopolymers and copolymers are particularly preferred for the purposes of the invention because the resulting preparations according to the invention undergo little or no change in viscosity, even during long-term storage at elevated temperatures.

Other Auxiliaries and Additives (Optional)

The compositions according to the invention may contain other auxiliaries and additives according to the particular application envisaged, including for example fats and waxes, pearlizing waxes, superfatting agents, stabilizers, cationic, zwitterionic or amphoteric polymers, biogenic agents, film formers, swelling agents, hydrotropes, preservatives, antidandruff agents, self-tanning agents, solubilizers, perfume oils, dyes, etc. which are mentioned by way of example in the following.

Fats and waxes in the context of the invention are understood to be any lipids of fat- or wax-like consistency which have a melting point above 20° C. These include, for example, the standard triacyl glycerols, i.e. the triple esters of fatty acids with glycerol which may be of vegetable or animal origin. They may also be mixed esters, i.e. triple esters of glycerol with various fatty acids, or mixtures of various glycerides, including mixtures of mono-, di- and triglycerides. So-called hardened fats and oils obtained by partial hydrogenation are also particularly suitable for the purposes of the invention. Vegetable hardened fats and oils are preferred, for example hardened castor oil, peanut oil, soybean oil, rape oil, rapeseed oil, cotton-seed oil, sunflower oil, palm oil, palm kernel oil, linseed oil, almond oil, corn oil, olive oil, sesame oil, cocoa butter and coconut oil. Oxidation-stable vegetable glycerides commercially available as Cegesofte and Novata® are particularly suitable.

Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Besides the fats, other suitable additives are fat-like substances, such as lecithins and phospholipids. Lecithins are glycerophospholipids which are formed from fatty acids, glycerol, phosphoric acid and choline by esterification and are also frequently referred to as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates). Sphingosines and sphingolipids are also suitable as fat-like substances.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially Lauron®; distearylether; fatty acids, such as stearic acid, C₁₂₋₂₂ hydroxyfatty acids, behenic acid, ring opening products of C₁₂₋₂₂ olefin epoxides with C₁₂₋₂₂ fatty alcohols and/or polyols containing 2 to 15 carbon atoms and 2 to 10 hydroxyl groups and mixtures thereof.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Metal salts of fatty acids such as, for example, magnesium, aluminum and/or zinc stearate or ricinoleate may be used as stabilizers.

Suitable cationic polymers, which further optimize the sensory profile of the compositions according to the invention and give the skin a feeling of softness, are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grunau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquate 550, Chemviron), polyaminopolyamides and crosslinked water-soluble polymers thereof, cationic chitin derivatives such as, for example, quatemized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable silicone compounds were mentioned above in connection with the oil components. Besides dimethyl polysiloxanes, methylphenyl polysiloxanes and cyclic silicones, other suitable silicone compounds are amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and silicon dioxide or hydrogenated silicates.

Biogenic agents suitable for the purposes of the invention are, for example, tocopherol, tocopherol acetate, tocopherol palmitate, ascorbic acid, glucans, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes. Active components such as these are used as radical traps in sun protection formulations and serve to regenerate the skin.

So-called film formers, which lead to a further improvement in the sensory profile of the preparations according to the invention, are, for example, chitosan, microcrystalline chitosan, quaternized chitosan, collagen, hyaluronic acid and salts thereof and similar compounds and the polyvinyl pyrrolidones, vinyl pyrrolidone/vinyl acetate copolymers, polymers of the acrylic acid series and quaternary cellulose derivatives.

Suitable antidandruff agents are Pirocton Olamin (1-hydroxy-4-methyl-6-(2,4,4-trimethylpentyl)-2-(1H )-pyridinone monoethanolamine salt), Baypival® (Climbazole), Ketoconazol® (4-acetyl-l-{4-[2-(2,4-dichloro-phenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminum pyrithione and magnesium pyrithione/dipyrithione magnesium sulfate.

A suitable self-tanning agent is, for example, dihydroxyacetone. Suitable tyrosinase inhibitors, which prevent the formation of melanin and are used in depigmenting agents, are, for example, arbutin, ferulic acid, koji acid, coumaric acid and ascorbic acid (vitamin C).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
short-chain alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetik-verordnung (“Cosmetics Directive”).

Suitable perfume oils are natural, vegetable and animal and also synthetic perfumes or mixtures thereof. Natural perfumes are obtained inter alia by extraction of flowers, stems and leaves, fruits, fruit peel, roots and resins of plants. Animal raw materials, for example civet and beaver, may also be used. Typical synthetic perfume compounds are products of the ester, ether, aldehyde, ketone, alcohol and hydrocarbon type. Mixtures of various perfumes, which together produce an attractive perfume note, are preferably used.

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106.

The total percentage content of auxiliaries and additives may be from 1 to 50% by weight and is preferably from 5 to 40% by weight, based on the preparations according to the invention. The preparations according to the invention may be produced by standard hot or cold processes and are preferably produced by the phase inversion temperature method.

### EXAMPLES

A general synthesis procedure and the components used and the quantities in which they are used are described in the following. The synthesis is normally carried out in the presence of sulfosucinic acid as catalyst.

General Synthesis Procedure

The diol (Pripol® 2033) was introduced into a dry reaction vessel and sulfosuccinic acid was added with stirring. The reactor was then heated to at most 200° C. under nitrogen and normal pressure. The reaction was continued at that temperature until the quantity of water calculated for a certain average molecular weight had separated. The reaction mixture was then cooled to 90° C. and sodium methanolate was added for neutralization. The crude product was dried in vacuo for 1 hour and packed in containers while still warm.

Polvalkylene Ether I Based on Pripol® 2033:

Pripol ® 2033 [dimer diol],

| | |
|---|---|
| OH value = 209 | 2148 g (4 mol) |
| sulfosuccinic acid | 14 g (70% solution in water; 0.05 mol) |
| sodium methanolate | 0.295 kg (30% solution in MeOH; 0.05 mol) |

Product Data Polyalkylene Ether l:
pale yellowish liquid; OH value: 81; acid value: <0.1, viscosity: ca. 16,000 mPa.s (Brookfield RVF, spindle 5, 10 rpm, 23° C.); quantity of water separated: 45.5 ml; average molecular weight: 1400.

Polyalkylene Ether 11 Based on Sovermol® 908:

Solvermol ® 908 [dimer diol],

| | |
|---|---|
| OH value = 209 | 2125 g (4 mol) |
| sulfosuccinic acid | 14 g (70% solution in water; 0.05 mol) |
| sodium methanolate | 0.295 kg (30% solution in MeOH; 0.05 mol) |

Product Data Polyalkylene Ether II:
OH value: 79; acid value: <0.1, viscosity: ca. 16,000 mPa.s (Brookfield RVF, spindle 5, 10 rpm, 23° C.); quantity of water separated: 45.0 ml; average molecular weight: 1400.

Polyalkylene Ether III Based on Pripol® 2033:

Pripol ® 2033 [dimer diol],

| | |
|---|---|
| OH value = 209 | 2034 g (3.77 mol) |
| sulfosuccinic acid | 14 g (70% solution in water; 0.05 mol) |
| sodiu methanolate | 0.295 kg (30% solution in MeOH; 0.05 mol) |

Product Data Polyalkylene Ether III:
pale yellowish liquid; OH value: 104; acid value: <0.2, viscosity: ca. 8,000 mPa.s (Brookfield RVF, spindle 5, 10 rpm, 23° C.); quantity of water separated: 38.0 ml; average molecular weight: 1000.

Polyalkylene Ether IV Based on Pripol® 2033:

Pripol ® 2033 [dimer diol],

| | |
|---|---|
| OH value = 209 | 2148 g (4 mol) |
| sulfosuccinic acid | 14 g (70% solution in water; 0.05 mol) |
| sodiu methanolate | 0.295 kg (30% solution in MeOH; 0.05 mol) |

Product Data Polyalkylene Ether IV:
pale yellowish liquid; OH value: 56; acid value: <0.2, viscosity: ca. 30,000 mPa.s (Brookfield RVF, spindle 5, 10 rpm, 23° C.); quantity of water separated: 73.0 ml; average molecular weight: 2000.

The polyalkylene ethers were incorporated in basic formulations and the water resistance of the preparations according to the invention was determined. To determine the water resistance of the preparations according to the invention, a defined quantity of the preparations (cf. Table 1) was applied to a suitable carrier material and was “watered” to predetermined criteria in a glass beaker, the water being agitated by a magnetic stirrer. The SPF (sun protection factor) was determined before and after the treatment with water using a UV 1000S Labsphere Ultraviolet Transmittance Analyzer.

The sensory evaluation was conducted by a panel of ten trained volunteers who awarded scores of (1)=very good to (6)=unsatisfactory. The results represent the mean values of three measurements.

Water Resistance

carrier material: Vitro-Skin N19, manuf.: IMS (4×3 cm) on slide frames
quantity applied: 2 mg/cm²
drying time before 1st measurement: 15 mins., temp. 30° C.
water temperature: 23° C. (16° d)
pH value water: 7.0±0.5
water volume: 400 ml
stirring speed: 300 r.p.m. (magnetic stirrer)
watering time: 2×20 mins. with an interval of 20 mins.
drying time before 2nd measurement: 15 mins., temp. 30° C.

The results are set out in Table 1. Examples 1 to 4 correspond to the invention; Examples C1 and C2 are intended for comparison. Unless otherwise indicated, the quantities mentioned in the following Examples represent % by weight of the commercially available substances in the composition as a whole.

TABLE 1a
Basic formulations of sun protection formulations; water resistance
and sensory profile

| Composition/Performance | 1 | 2 | 3 | 4 | C1 | C2 |
|---|---|---|---|---|---|---|
| Eumulgin ® VL 75 | 4.0 | 4.0 | 4.0 | 4.0 | 4.0 | 4.0 |
| Myritol ® 331 | 7.0 | 7.0 | 7.0 | 7.0 | 7.0 | 7.0 |
| Cetiol ® OE | 6.0 | 6.0 | 6.0 | 6.0 | 6.0 | 6.0 |
| Eutanol ® G 16 | 3.0 | 3.0 | 3.0 | 3.0 | 3.0 | 3.0 |
| Polyalkylene ether I, MW 1400 | 4.0 | | | 2.0 | | |
| Polyalkylene ether III, MW 1000 | | 4.0 | | | | |
| Polyalkylene ether IV, MW 1000 | | | 4.0 | | | |
| Antaron ® V 220 | | | | 2.0 | 4.0 | |
| Antaron ® V 216 | | | | | | 4.0 |
| Neo Heliopan ® AV | 7.5 | 7.5 | 7.5 | 7.5 | 7.5 | 7.5 |
| Parsol ® 1789 | 2.0 | 2.0 | 2.0 | 2.0 | 2.0 | 2.0 |
| Carbopol ® 2984 | 0.2 | 0.2 | 0.2 | 0.2 | 0.2 | 0.2 |
| Glycerin | 5.0 | 5.0 | 5.0 | 5.0 | 5.0 | 5.0 |
| Water/NaOH/preservative | to 100/pH = 7/q.s | | | | | |
| In-vitro Sun Protection Factor (SPF) | | | | | | |
| Before treatment with water | 15 | 15 | 15 | 15 | 15 | 15 |
| After treatment with water | 14 | 13 | 14 | 13 | 9 | 10 |
| Difference (% -rel.) | 93 | 87 | 933 | 87 | 60 | 66 |
| Sensory evaluation | | | | | | |
| Absorption | 1 | 1 | 1 | 1 | 5 | 4 |
| Smoothness | 1 | 1 | 2 | 1 | 4 | 4 |
| Tackiness | 1 | 1 | 1 | 2 | 6 | 5 |

Comparison Examples C1 and C2, which contain Antaron® V220 and Antaron® V 216 instead of the polyalkylene ethers, show distinctly reduced water resistance and are inferior in their sensory properties.

TABLE 2
O/W sunprotection emulsions
Quantities represent % by weight of the commercially available
substances in the composition as a whole.

| Component | 5 | 6 | 7 | 8 | 9 | 10 | 11 | 12 | 13 | 14 | 15 |
|---|---|---|---|---|---|---|---|---|---|---|---|
| L = Lotion, C = Cream, S = Spray | L | C | S | L | C | L | L | C | L | C | L |
| Eumulgin ® VL 75 | | | | | | 4 | 4 | 2 | | | 2 |
| Eumulgin ® B2 | 2 | | | | | | | | | | |
| Tween ® 60 | | | | 1 | | | | | | | |
| Myrj ® 51 | | 3 | | 2 | | | | | | | |
| Cutina ® E 24 | 1 | | | 1 | | | | | | | |
| Hostaphat ® KL 340 N | | | | | | | | | 2 | | |
| Lanette ® E | | | 0.5 | | | | | 0.5 | | | |
| Amphisol ® K | | | 1 | | | 1 | | 0.5 | | 1 | |
| Sodium stearate | | | | | | | 1 | | | | 2 |
| Emulgade ® PL 68/50 | | | 1 | | 5 | | | | | 4 | |
| Tego ® Care 450 | | | | | | | | | | 3 | |
| Cutina ® MD | 2 | | | 6 | | | 4 | | | 6 | |
| Lanette ® 14 | 1 | | | 1 | | | | 2 | | | 4 |
| Lanette ® O | 1 | 6 | | | 5 | 2 | | 2 | | | |
| Polyalkylene ether I | 2 | | 4 | | | 2 | 2 | | 2 | | |
| Polyalkylene ether III | | 2 | | 1 | | | | | | | 1 |
| Polyalkylene ether IV | | | | | 2 | | | 3 | | 2 | |
| Emery ® 1780 | | | | | 0.5 | 0.5 | | | | | |
| Lanolin, anhydrous, USP | | | | | | | 5 | | | | |
| Myritol ® PC | | | | | 5 | | | | | | |
| Myritol ® 331 | 5 | | 8 | | | 6 | | 10 | | 2 | |
| Finsolv ® TN | | | 1 | | | | | 1 | 8 | | |
| Cetiol ® CC | | 2 | 5 | | | 4 | 4 | 2 | | 2 | 3 |
| Cetiol ® OE | | | 3 | | | | | | 2 | 3 | |
| Dow Corning DC ® 244 | 4 | | 1 | | 5 | | | 2 | | | 2 |
| Dow Corning DC ® 2502 | | 1 | | | 2 | | | | | | |
| Squatol ® S | | | | | | | 4 | | | | |
| Silikonöl Wacker AK ® 350 | | 2 | | | | | | | | | |
| Cetiol ® 868 | | | | | 2 | | 4 | | | | 7 |
| Cetiol ® J 600 | | | | | 3 | 2 | | | | 5 | |
| Mineral oil | | | | 9 | | | | | | | |
| Cetiol ® B | | | 1 | | | | | | | 2 | |
| Eutanol ® G | | | | | | | | | | | |
| Eutanol ® G 16 | | | | | | | | | | | |
| Cetiol ® PGL | | 5 | | | | | | | | 5 | |
| TABLE 2 ctd. | | | | | | | | | | | |
| O/W sun protection emulsions | | | | | | | | | | | |
| Component | 5 | 6 | 7 | 8 | 9 | 10 | 11 | 12 | 13 | 14 | 15 |
| Almond oil | | | 2 | | | | 1 | | | | |
| Photonyl ® LS | | | | 2 | | | | | | 2 | |
| Panthenol | 1 | | | | | | | | | | |
| Bisabolol | 0.2 | | | | | | | | | | |
| Tocopherol/Tocopheryl acetate | 1 | | | | | | | | | | |
| Photonyl ® LS | | | | | | | | | | | |
| Neo Heliopan ® Hydro (Na salt) | 2 | | 2.2 | | 3 | 3 | | | | | 2 |
| Neo Heliopan ® 303 | 3 | 5 | 9 | 4 | | | | | | | |
| Neo Heliopan ® BB | | | | | 1 | | | | | | 2 |
| Neo Heliopan ® MBC | 2 | | | 3 | | 2 | 2 | 2 | | | 1 |
| Neo Heliopan ® OS | | | | | | | | | 10 | 7 | |
| Neo Heliopan ® E 1000 | | 7.5 | | 6 | | | | | | | 6 |
| Neo Heliopan ® AV | | | 7.5 | | | 7.5 | 4 | 5 | | | |
| Uvinul ® T 150 | 2 | | | 2 | 2.5 | | | 1 | 1.5 | 2 | |
| Parsol ® 1789 | | 1 | 1 | | | | 2 | | 2 | 2 | |
| Zinc oxide NDM | 10 | | 5 | | | 10 | | 3 | | 5 | 4 |
| Eusolex ® T 2000 | | | | | 5 | | 3 | 3 | | | 4 |
| Veegum ® Ultra | | | 0.75 | | | | | 1 | 1 | | |
| Keltrol ® T | | | 0.25 | | | | | 0.5 | 0.5 | | |
| Carbopol ® 980 | | 0.5 | | 0.2 | 0.2 | 0.2 | | 0.5 | 0.1 | 0.3 | 0.2 |
| Ethanol | | | | | | | | | | 10 | |
| Butylene glycol | | 2 | | 4 | 3 | | 2 | 5 | 2 | | 2 |
| Glycerin | 5 | 5 | 5 | | 3 | 3 | 2 | | 4 | | 3 |

| | |
|---|---|
| Preservative, NaOH, Water | q.s., to 100 |

TABLE 3
O/W sun protection emulsions
Quantities represent % by weight of the commercially available
substances in the composition as a whole.

| Component | 16 | 17 | 18 | 19 | 20 | 21 | 22 | 23 | 24 | 25 | 26 |
|---|---|---|---|---|---|---|---|---|---|---|---|
| L = Lotion, C = Cream, S = Spray | L | L | L | C | L | C | S | C | C | L | L |
| Eumulgin ® VL 75 | 4 | 3 | 4.5 | | 3 | | | | 4 | | |
| Eumulgin ® B2 | | | | | | | | | | 1 | |
| Tween ® 60 | | | | | | | | | | 1 | |
| Myrj ® 51 | | | | | | | | | | | |
| Cutina ® E 24 | | | | 2 | | | | | | | |
| Hostaphat ® KL 340 N | | | | | | | | | | 0.5 | |
| Lanette ® E | 0.5 | | 0.5 | 0.5 | | | 0.1 | | 0.5 | | |
| Amphisol ® K | 0.5 | | | | | 1 | 1 | 1 | | | |
| Sodium stearate | | | | | 1 | | | | | | |
| Emulgade ® PL 68/50 | | 6 | | | | 4.5 | 1 | 5 | | | |
| Tego ® Care 450 | 1 | | | | | | | | 4 | | |
| Cutina ® MD | 1 | | | 8 | 6 | 1 | | | | 4 | 1 |
| Lanette ® 14 | | 2 | | | | | | 2 | | 1 | |
| Lanette ® O | | | | 2 | | | | | 1 | 1 | |
| Polyalkylene ether I | 4 | | 4 | | | | 2 | | | | 3 |
| Polyalkylene ether III | | | | 1 | | | | | 2 | 4 | |
| Polyalkylene ether IV | | 2 | | | 2 | 4 | | 2 | | | |
| Emery ® 1780 | | | | | | | | | | | |
| Lanolin, anhydrous, USP | | | | | | | | | | | |
| Myritol ® PC | | | | | | | | | 5 | | |
| Myritol ® 331 | 12 | | 12 | | | 8 | 8 | | | 10 | 8 |
| Finsolv ® TN | | | | | 5 | | | 3 | 3 | | |
| Cetiol ® CC | 6 | | 6 | | | 5 | 5 | | | | |
| Cetiol ® OE | | | | | 2 | | | | | | 2 |
| Dow Corning DC ® 244 | | 2 | | | 1 | | | | | | |
| Dow Corning DC ® 2502 | | 1 | | | 1 | | | | | | |
| Ceraphyl ® 45 | | | | | | | | | | 2 | 2 |
| Silikonöl Wacker AK ® 350 | | | | | 1 | | | | | | |
| Cetiol ® 868 | | 2 | | | | | | | | | |
| Cetiol ® J 600 | | 2 | | | | | | | | | |
| Mineral oil | | | | 10 | | | | | | | |
| Cetiol ® B | 4 | | 4 | | | | | 4 | | | |
| Eutanol ® G | | 3 | | | | 3 | | | | | |
| Eutanol ® G 16 S | 10 | | | | | | | | | | |
| Cetiol ® PGL | | | | | | | | | 2 | | |
| Photonyl ® LS | | | | | | | | | | 2 | |
| Panthenol | 1 | | | | | | | | | | |
| Bisabolol | 0.2 | | | | | | | | | | |
| Tocopherol/Tocopheryl acetate | 1 | | | | | | | | | | |
| Neo Heliopan ® Hydro (Na salt) | | | | | | | | | | 3 | |
| Eusolex ® OCR | 6 | | 9 | | 5 | 7 | 9 | 3 | 4 | | 7 |
| Neo Heliopan ® BB | | | | | | 1 | | 1 | 1 | | 1 |
| Neo Heliopan ® MBC | | 2 | | 1 | | 2 | | 3 | 1 | | 3 |
| Neo Heliopan ® OS | 2 | | | | | | | | 7 | | |
| Neo Heliopan ® E1000 | | 4 | | | | | | 5 | | | |
| Neo Heliopan ® AV | | 4 | 7.5 | 5 | | 3 | | 5 | 4 | 7.5 | 3 |
| Uvinul ® T 150 | 1 | | 1 | | 1 | 1 | 1 | | 1.3 | 1 | 1 |
| Parsol ® 1789 | 1 | | | | | 1 | | | 2 | | 1 |
| Z-Cote ® HP 1 | 7 | 2 | 5 | | | 7 | 5 | | 6 | 2 | 2 |
| Eusolex ® T 2000 | 5 | 2 | | | 10 | | | 10 | | 2 | 2 |
| Veegum ® Ultra | 1.5 | | 1.5 | | | 1.5 | 1.2 | | 1 | | |
| Keltrol ® T | 0.5 | | 0.5 | | | 0.5 | 0.4 | | 0.5 | | |
| Pemulen ® TR 2 | | 0.3 | | 0.3 | | | 0.1 | 0.2 | | | 0.3 |
| Ethanol | | 5 | | 8 | | | | | | | |
| Butylene glycol | 1 | | | 3 | 3 | | | | | 8 | 1 |
| Glycerin | 2 | 4 | 3 | 3 | | 3 | 3 | 3 | 5 | | 3 |

| | |
|---|---|
| Water/preservative/NaOH | to 100/q.s./q.s |

TABLE 4
W/O sun protection emulsions
Quantities represent % by weight of the commercially available
substances in the composition as a whole.

| Component | 27 | 28 | 29 | 30 | 31 | 32 | 33 | 34 | 35 | 36 | 37 |
|---|---|---|---|---|---|---|---|---|---|---|---|
| L = Lotion; C = Cream | C | L | C | L | C | L | L | L | L | C | C |
| Dehymuls ® PGPH | 4 | 2 | 1 | 3 | 3 | 1 | 1 | 2 | 2 | 4 | 1 |
| Monomuls ® 90-O18 | | | 2 | | | | | | | | |
| Lameform ® TGI | 2 | | 4 | | 3 | | | | | 1 | 3 |
| Abil ® EM 90 | | | | | | | 4 | | | | |
| Glucate ® DO | | | | | | | | | | | 3 |
| Isolan ® PDI | | | | | | 4 | | 2 | | | |
| Arlacel ® 83 | | | | 2 | | | | | | | |
| Elfacos ® ST9 | | | | | | | | | 2 | | |
| Elfacos ® ST37 | | | | | | | | | | | |
| Arlacel ® P 135 | | 2 | | | | | | | | | |
| Dehymuls ® HRE 7 | | | | | | | | | | | |
| Zinc stearate | 1 | | | 1 | 1 | | | 1 | | 1 | |
| Microcrystalline wax | | | 5 | | | 2 | | | | | 5 |
| Beeswax | 1 | | | 1 | | | | 5 | | 7 | |
| Tego ® Care CG | | | | | 1 | | | | | | .5 |
| Prisorine ® 3505 | 1 | | 1 | 1 | | 1 | 1 | | | | 1 |
| Polyalkylene ether I | 3 | 4 | | 4 | | 2 | | | 1 | 1 | |
| Polyalkylene ether III | | | | | | | | 2 | 1 | | |
| Polyalkylene ether IV | | | 2 | | 4 | | 2 | | 1 | | 3 |
| Emery ® 1780 | | | 5 | | | | | | | 4 | |
| Wool wax alcohol, anhydrous, USP | | | | | | | | | | | 1 |
| Myritol ® PC | | | | | 3 | | | 4 | | | |
| Myritol ® 331 | 10 | | | | 3 | 6 | | | | | 8 |
| Finsolv ® TN | | | | 5 | | | 5 | | | | |
| Cetiol ® CC | 12 | 22 | | | | 2 | | | 2 | | 5 |
| Cetiol ® OE | | | | | 4 | | 5 | | 4 | 2 | |
| Dow Corning DC ® 244 | | | | | | | 2 | | | | |
| Dow Corning DC ® 2502 | | | 1 | | 2 | | | | | | |
| Prisorine ® 3758 | | | | | | | | | | 2 | |
| Silikonöl Wacker AK ® 350 | | | | 4 | | | | 3 | | | |
| Cetiol ® 868 | | | | | | | | | | 2 | |
| Eutanol ® G 16 | | 3 | | | | | | | | | |
| Eutanol ® G 16S | | | | | | | | | | | |
| Cetiol ® J 600 | | | 4 | | | 2 | | | | | |
| Ceraphyl ® 45 | | | | 2 | | | | 2 | | 6 | |
| Mineral oil | | | | | 4 | | | | | | |
| Cetiol ® B | | | 2 | 4 | | | | | | 3 | |
| Eutanol ® G | | | 3 | | | | | 8 | | | |
| Cetiol ® PGL | | 11 | | | | 4 | | | 9 | | |
| Almond oil | | | | | 1 | | 5 | | | | |
| Photonyl ® LS | | 2 | | 1 | | | | | 4 | | |
| Panthenol | 1.0 | | | | | | | | | | |
| Bisabolol | 0.2 | | | | | | | | | | |
| Tocopherol/Tocopheryl acetate | 1.0 | | | | | | | | | | |
| Magnesium sulfate × 7 water | 1 | | | | | | | | | | |
| Neo Heliopan ® Hydro (Na salt) | | 2 | | 3 | 1 | | | 2 | 1 | 1 | |
| Neo Heliopan ® 303 | | | | 2 | 4 | | | 2 | | 6 | |
| Neo Heliopan ® BB | | 4 | 2 | | | | 2 | | | | |
| Neo Heliopan ® MBC | | | | 2 | 2 | | | 4 | | 3 | |
| Neo Heliopan ® OS | | | | | | | | | | | |
| Neo Heliopan ® E 1000 | | | | | | | | | 5 | | |
| Neo Heliopan ® AV | | 3 | 6 | 6 | 3 | 7.5 | 7.5 | | 5 | | 7.5 |
| Uvinul ® T 150 | | 1 | | 2 | 2.5 | | | 1 | | 2 | |
| Parsol ® 1789 | | 2 | | | 1 | | | 1 | | 2 | |
| Zinc oxide NDM | | | | 2 | 5 | 6 | 4 | | 3 | 2 | 15 |
| Eusolex ® T 2000 | 15 | | 10 | 2 | 5 | 6 | 4 | | 3 | 2 | |
| Ethanol | | | | | | | | | | 8 | |
| Butylene glycol | | | 2 | 6 | | | 2 | 5 | | | 2 |
| Glycerin | 5 | 3 | 3 | | 5 | 3 | 2 | | 10 | 4 | |

| | |
|---|---|
| Water, preservative | to 100, q.s. |

TABLE 5
W/O sun protection emulsions
Quantities represent % by weight of the commercially available
substances in the composition as a whole.

| Component | 38 | 39 | 40 | 41 | 42 | 43 | 44 | 45 | 46 | 47 | 48 |
|---|---|---|---|---|---|---|---|---|---|---|---|
| L = Lotion; C = Cream | L | C | L | L | C | L | L | L | L | C | C |
| Dehymuls ® PGPH | 3 | 1 | 5 | 1 | 1 | 3 | 2 | 4 | 0.5 | 1 | 4 |
| Monomuls ® 90-O18 | | 1 | | | | | | | | | |
| Lameform ® TGI | | | | | 4 | | | 1 | | 3 | 1 |
| Abil ® EM 90 | | | | 1 | | | | | | 2 | |
| Glucate ® DO | | | | 3 | | | | | 2 | | |
| Isolan ® PDI | | 3 | | | | | 4 | | | | |
| Arlacel ® 83 | | | | | | 3 | | | | | |
| Elfacos ® ST9 | | | | | | | | | | | 2 |
| Elfacos ® ST37 | 2 | | | | | | | | | | |
| Arlacel ® P 135 | | | | | | 3 | | | | | |
| Dehymuls ® HRE 7 | | | | | | | | | 4 | | |
| Zinc stearate | | 2 | 2 | 1 | 1 | | | 1 | 1 | | |
| Microcrystalline wax | | | | | 4 | | 1 | | | 4 | |
| Beeswax | | 4 | | 2 | | | 1 | | 2 | | 1 |
| Tego ® Care CG | | | | | | | | | | | |
| Isostearic acid | 1 | 1 | | | | | 1 | 1 | | 1 | 1 |
| Polyalkylene ether I | 2 | 4 | | | 2 | | | | 3 | | 4 |
| Polyalkylene ether III | | | 3 | | | | 1 | | | | |
| Polyalkylene ether IV | | | | 3 | | 2 | | 3 | | 1 | |
| Emery ® 1780 | | 7 | 3 | | | | | | | | |
| Wool wax alcohol, anhydrous, USP | | | | | | | | | | | |
| Myritol ® PC | | | | | | | | | | | |
| Myritol ® 331 | 4 | 2 | 3 | | 5 | | | 8 | 5 | 4 | |
| Finsolv ® TN | | 5 | 5 | | | 7 | | | | | |
| Cetiol ® CC | 3 | 1 | | | | | 3 | 16 | | | 12 |
| Cetiol ® OE | | 3 | | 2 | | | 3 | | | | |
| Dow Corning DC ® 244 | | 4 | | 2 | | | | | | | |
| Dow Corning DC ® 2502 | | | | 1 | | | | | | | |
| Prisorine ® 3578 | | 1 | | | | | | | | | |
| Silikonöl Wacker AK ® 350 | | | | 1 | | | | | | | |
| Cetiol ® 868 | | | | | | | | | | | |
| Eutanol ® G 16 | | | | | | | | | | | 3 |
| Eutanol ® G 16S | | | | | | | | | | | 7 |
| Cetiol ® J 600 | | | | 3 | | | | | | | |
| Ceraphyl ® 45 | | | | 1 | | | | | 5 | 4 | |
| Mineral oil | | | | | | | 9 | | | | |
| Cetiol ® B | | | | | 3 | 3 | | 2 | 2 | | |
| Eutanol ® G | | | | 2 | | | | | 5 | | |
| Cetiol ® PGL | | | | | | | | 2 | | | |
| Almond oil | | | 2 | | | | | | | | |
| Photonyl ® LS | | | | | | | 3 | | | | 2 |
| Panthenol | 1.0 | | | | | | | | | | |
| Bisabolol | 0.2 | | | | | | | | | | |
| Tocopherol/Tocopheryl acetate | 1.0 | | | | | | | | | | |
| Magnesium sulfate × 7 water | 1 | | | | | | | | | | |
| Neo Heliopan ® Hydro (Na salt) | | 4 | | 1 | 1 | | | 4 | | | |
| Neo Heliopan ® 303 | 6 | 2 | | | 2 | | | | 6 | | |
| Neo Heliopan ® BB | | 2 | | 2 | | 2 | | | | | |
| Neo Heliopan ® MBC | 2 | | | 1 | 3 | | 4 | | 2 | | |
| Neo Heliopan ® OS | | | | | 4 | | 8 | | | | |
| Neo Heliopan ® E 1000 | | | 5 | | | | | | | 5 | |
| Neo Heliopan ® AV | | 5 | 5 | 6 | 4 | 7.5 | | | | 5 | |
| Uvinul ® T 150 | 1 | 1 | | 2 | 2 | | | | 3 | 2 | |
| Parsol ® 1789 | | 1 | 1 | 1 | | | 1 | | 0.5 | | |
| Z-Cote ® HP 1 | 4 | 10 | | 2 | 2 | | 8 | 5 | 5 | | 5 |
| Titanium dioxide T 805 | | | | 2 | 2 | 10 | | 7 | 7 | 4 | 7 |
| Ethanol | | | | 8 | | 10 | | | | | |
| Butylene glycol | 5 | 1 | | 3 | 3 | | | | 8 | 2 | |
| Glycerin | | | 6 | 2 | | | 5 | 5 | | 3 | 5 |

| | |
|---|---|
| Water, preservative | to 100, q.s. |

TABLE 6
W/O care emulsions
Quantities represent % by weight of the commercially available
substances in the composition as a whole.

| Componente | 49 | 50 | 51 | 52 | 53 | 54 | 55 | 56 | 57 | 58 | 59 |
|---|---|---|---|---|---|---|---|---|---|---|---|
| L = Lotion. C = Cream | C | L | C | L | C | L | L | L | C | C | C |
| Polyalkylene ether I | 1 | | | 1 | | 1 | 1 | 1 | | 2 | |
| Polyalkylene ether III | | 1 | | | | | | | 2 | | |
| Polyalkylene ether IV | | | 2 | | 1 | | | | | | 1 |
| Dehymuls ® PGPH | 1 | 3 | 1 | 2 | 3 | 1 | 1 | 2 | 1 | 1 | 1 |
| Monomuls ® 90-O18 | 2 | | | | | | | | 2 | | 2 |
| Lameform ® TGI | 4 | 1 | | | 3 | | | 1 | 4 | 3 | 3 |
| Abil ® EM 90 | | | | | | | 4 | | | | |
| Isolan ® PDI | | | | | | 4 | | | | | |
| Glucate ® DO | | | | 5 | | | | | | | |
| Arlacel ® 83 | | | 5 | | | | | | | | |
| Dehymuls ® FCE | | | | | | | | | | | |
| Dehymuls ® HRE 7 | | | | | | | | 4 | | 1 | |
| Zinc stearate | 2 | 1 | | 1 | 1 | | | 1 | 1 | 1 | |
| Microcrystalline wax | | | 5 | | | 2 | | | | | 5 |
| Beeswax | 4 | | | 1 | | | | 1 | 4 | 7 | |
| Tego Care ® CG | | | | | 1 | | | | | | 0.5 |
| Prisorine ® 3505 | | | 1 | 1 | | 1 | 1 | | | | 1 |
| Dry Flo ® Plus | | | | | | | | | | | |
| SFE 839 | | | | | | | 3 | | | | |
| Emery ® 1780 | 1 | | | | | | | | | | 1 |
| Lanolin; anhydrous USP | | | 5 | | | | | | | 4 | |
| Cegesoft ® C 17 | | | 3 | | | | | | | 1 | |
| Myritol ® PC | | | | | | 2 | | 4 | | | |
| Myritol ® 331 | 6 | | | | 2 | 6 | 2 | | | | 8 |
| Finsolv ® TN | | | | 5 | | 2 | 5 | | | | |
| Cetiol ® A | | 6 | | | | 4 | | | | | |
| Cetiol ® CC | | 8 | | | 2 | 2 | 2 | | | | 5 |
| Cetiol ® SN | | 5 | | | | | | 3 | | | |
| Cetiol ® OE | 3 | | | | 4 | | 2 | | 4 | 2 | |
| Dow Corning DC ® 244 | | | | | 1 | | 2 | | | | |
| Dow Corning DC ® 2502 | | | 1 | | 2 | | | | | | |
| Prisorine ® 3758 | | | | | 3 | | | | | | |
| Silikonöl Wacker AK ® 350 | | | | 4 | | | | 3 | | | |
| Cetiol ® 868 | | | | | | | | | | 2 | 7 |
| Cetiol ® J 600 | | | 4 | | | 2 | | | | | |
| Ceraphyl ® 45 | | | | 2 | | | | 2 | | 6 | |
| Mineral oil | | | | | 4 | | | | | | |
| Cetiol ® B | | | 2 | 4 | | | | | | 3 | |
| Eutanol ® G 16 | | 1 | | | | | | | | 3 | |
| Eutanol ® G | | | 3 | | | | | 8 | | | |
| Cetiol ® PGL | | | | | | 4 | | | 9 | | |
| Almond oil | | | | | 1 | | 5 | | | | |
| Insect Repellent ® 3535 | 2 | | | | | | | | | | |
| N,N-Diethyl-m-toluamide | | | | 3 | | | | 5 | | | |
| Photonyl ® LS | 2 | 2 | | | | | | | | | |
| Panthenol | 1.0 | | | | | | | | | | |
| Bisabolol | 0.2 | | | | | | | | | | |
| Tocopherol/Tocopheryl Acetate | 1.0 | | | | | | | | | | |
| Magnesium sulfate × 7 water | 1 | | | | | | | | | | |
| Bentone ® 38 | | | | | 1 | | | | | | |
| Propylene carbonate | | | | | 0.5 | | | | | | |
| Ethanol | | | | | | | | | | 8 | |
| Butylene Glycol | | | 2 | 6 | | | 2 | 5 | | | 2 |
| Glycerin | 5 | 3 | 3 | | 5 | 3 | 2 | | 10 | 4 | |

| | |
|---|---|
| Water, preservative | to 100, q.s. |

TABLE 7
W/O care emulsions
Quantities represent % by weight of the commercially available
substances in the composition as a whole.

| Componente | 60 | 61 | 62 | 63 | 64 | 65 | 66 | 67 | 68 | 69 | 70 |
|---|---|---|---|---|---|---|---|---|---|---|---|
| L = Lotion, C = Cream | L | C | L | L | C | L | L | L | L | C | C |
| Polyalkylene ether I | 1 | | 1 | | 1 | | 1 | | | 1 | |
| Polyalkylene ether III | | 3 | | | | | | 1 | | | |
| Polyalkylene ether IV | | | | 2 | | 2 | | | 1 | | 3 |
| Dehymuls ® PGPH | 3 | 1 | 5 | 1 | 1 | 3 | 3 | 4 | 1 | 1 | 1 |
| Monomuls ® 90-O18 | | 1 | | 1 | | | | | | | |
| Lameform ® TGI | | | | | 4 | | | 1 | | 3 | |
| Abil ® EM 90 | | | | 3 | | | | | | 2 | |
| Isolan ® PDI | | 3 | | | | | | | | | 4 |
| Glucate ® DO | 1 | | | | | | | | | | |
| Arlacel ® 83 | | | | | | 3 | | | | | |
| Dehymuls ® FCE | | | | | 4 | | 1 | | | | |
| Dehymuls ® HRE 7 | | | | | | | | | 7 | | |
| Zinc stearate | | 2 | 2 | 1 | 1 | 1 | | 1 | 1 | | 1 |
| Microcryatalline wax | | | | | 4 | | 1 | | | 4 | |
| Beeswax | | 4 | | 2 | | 2 | 1 | 1 | 2 | | 5 |
| Tego ® Care CG | | | | | | | | | | | |
| Prisorine ® 3505 | 1 | 1 | | | | | 1 | 1 | | 1 | 1 |
| Dry Flo ® Plus | 1 | | | | | | | | | | |
| SFE ® 839 | | 5 | | | | 4 | | | | | |
| Emery ® 1780 | | | | | | | | | | | |
| Lanolin anhydrous USP | | 7 | 3 | | | | | | | | |
| Cegesoft ® C 17 | | 2 | | | | | | | | | |
| Myritol ® PC | | | | 8 | | | | | | | |
| Myritol ® 331 | 4 | | 3 | | 5 | 3 | | | 5 | 4 | |
| Finsolv ® TN | | | 5 | | | 7 | | | | | |
| Cetiol ® A | | | | | | | | 6 | | | |
| Cetiol ® CC | 3 | | | 6 | | 3 | 3 | | | 8 | |
| Cetiol ® SN | | | | | 5 | | | | | | |
| Cetiol ® OE | | 3 | | 2 | | | 3 | | | | 8 |
| Dow Corning ® DC 244 | | 4 | | 2 | | 2 | | | | | |
| Dow Corning ® DC 2502 | | | | 1 | | | | | | | |
| Prisorine ® 3758 | | | | | | 1 | | | | | |
| Silikonöl Wacker AK ® 350 | | | | 1 | | 1 | | 4 | | | |
| Cetiol ® 868 | | | | | | | | | | | 10 |
| Cetiol ® J 600 | 4 | | | 3 | | | | | | | |
| Ceraphyl ® 45 | | | | 1 | | | | | 5 | 4 | |
| Mineral oil | | | | | | | 9 | | | | |
| Cetiol ® B | | | | | 3 | 3 | | 2 | 2 | | |
| Eutanol ® G 16 | 1 | | | | | | | | | | |
| Eutanol ® G | | | | 2 | | | | | 5 | | |
| Cetiol ® PGL | | | 10 | | | | | 6 | | | 3 |
| Almond oil | | | 2 | | 5 | | 2 | | | | |
| Photonyl ® LS | | | | 2 | | | | | | | 2 |
| Panthenol | 1.0 | | | | | | | | | | |
| Bisabolol | 0.2 | | | | | | | | | | |
| Tocopherol/Tocopheryl acetate | 1.0 | | | | | | | | | | |
| Magnesium sulfate × 7 water | 1 | | | | | | | | | | |
| Bentone ® 38 | | | | | | 1 | | | | | |
| Propylene carbonate | | | | | | 0.5 | | | | | |
| Ethanol | | | | 8 | | 10 | | | | | |
| Butylene glycol | 5 | 1 | | 3 | 3 | | | | 8 | 2 | 1 |
| Glycerin | | | 6 | 2 | | | 5 | 5 | | 3 | 5 |

| | |
|---|---|
| Water, preservative | to 100, q.s. |

TABLE 8
O/W care emulsions
Quantities represent % by weight of the commercially available
substances in the composition as a whole.

| Component | 71 | 72 | 73 | 74 | 75 | 76 | 77 | 78 | 79 | 80 | 81 |
|---|---|---|---|---|---|---|---|---|---|---|---|
| L = Lotion, C = Cream | C | C | C | L | C | L | L | C | L | C | C |
| Polyalkylene ether I | 2 | 2 | 1 | | | | | 3 | 2 | | |
| Polyalkylene ether III | | | | 1 | | 1 | | | | | |
| Polyalkylene ether IV | | | | | 1 | | 2 | | | 2 | 1 |
| Eumulgin ® VL 75 | | | | | | 4 | 2 | | | | |
| Dehymuls ® PGPH | | 2 | | | | | | | | | |
| Generol ® R | | | 1 | | | | | | | | |
| Eumulgin ® B2 | | | 0.8 | | | | | | | | |
| Tween ® 60 | | | | 1 | | | | | | | |
| Cutina ® E 24 | | | 0.6 | 2 | | | | | | | |
| Hostaphat ® KL 340 N | | | | | | | | | 2 | | |
| Lanette ® E | | | | | | | | 1 | | | |
| Amphisol ® K | | 0.5 | | | | 1 | | | | 1 | 0.5 |
| Sodium stearate | | | | | 0.5 | | | | | | |
| Emulgade ® PL 68/50 | | 2.5 | | | | | | | | 4 | |
| Tego ® Care CG | | | | | | | | | | | 2 |
| Tego ® Care 450 | | | | | | | | 5 | | | |
| Cutina ® MD | | 1 | | 6 | 5 | | 4 | | | 6 | |
| Lanette ® 14 | | | | 1 | | | | 2 | | | 4 |
| Lanette ® O | 4.5 | | 4 | | 1 | 2 | | | | | 2 |
| Novata ® AB | | 1 | | | | | | | | | 1 |
| Emery ® 1780 | | | | | 0.5 | 0.5 | | | | | |
| Lanolin, anhydrous, USP | | | | | | | 5 | | | | |
| Cetiol ® SB 45 | | | 1.5 | | | | 2 | | | | |
| Cegesoft ® C 17 | | | | | | | | | | | |
| Myritol ® PC | | | | | 5 | | | | | | |
| Myritol ® 331 | 2 | 5 | 5 | | | 6 | | 12 | | | |
| Finsolv ® TN | | | 2 | | | 2 | | | 8 | | |
| Cetiol ® CC | 4 | 6 | | | | 4 | 4 | | | | 5 |
| Cetiol ® OE | | | | | | | | | 4 | 3 | |
| Dow Corning DC ® 245 | | | 2 | | 5 | 1 | | | | | |
| Dow Corning DC ® 2502 | | | | | 2 | 1 | | | | | |
| Prisorine ® 3758 | | | | | | 1 | | | | | |
| Silikonöl Wacker AK ® 350 | 0.5 | 0.5 | 0.5 | | | 1 | 4 | | | | |
| Cetiol ® 868 | | | | | 2 | | 4 | | | | |
| Cetiol ® J 600 | 2 | | 3 | | 3 | 2 | | | | 5 | |
| Ceraphyl ® 45 | | | | | | | 3 | | | | |
| Mineral oil | | | | 9 | | | | | | | |
| Cetiol ® SN | | | 5 | | | | | | | | |
| Cetiol ® B | | | | | | | | | | 2 | |
| Eutanol ® G | | 2 | | 3 | | | | | | | |
| Cetiol ® PGL | | | | | | | | | 5 | 5 | |
| Dry Flo ® Plus | 5 | | | | | | 1 | | | | |
| SFE 839 | 5 | | | | | | | | | | 2 |
| Almond oil | | | | | | | 1 | | | | |
| Insect Repellent ® 3535 | | 2 | 4 | | | 2 | | | | 3 | |
| N,N-Diethyl-m-toluamide | | 2 | | | | | | | | 3 | |
| Photonyl ® LS | 2 | 2 | | | | 2 | | | | | |
| Panthenol | 1 | | | | | | | | | | |
| Bisabolol | 0.2 | | | | | | | | | | |
| Tocopherol/Tocopheryl acetate | 1 | | | | | | | | | | |
| Veegum ® ultra | | | | | | | | | 1 | | |
| Keltrol ® T | | | 0.4 | | | | | | 0.5 | | |
| Pemulen ® TR 2 | 0.3 | | | | | | 0.1 | 0.3 | | | |
| Carbopol ® Ultrez 10 | | 0.3 | 0.3 | 0.2 | 0.2 | 0.2 | | | 0.1 | 0.3 | 0.2 |
| Ethanol | | | | | | | | | | 10 | |
| Butylene glycol | | | | 4 | 3 | | 2 | 5 | 2 | | |
| Glycerin | 2 | 5 | 5 | | 3 | 3 | 2 | | 4 | | 3 |

| | |
|---|---|
| Water, preservative, NaOH | to 100, q.s., pH 6.5-7.5 |

TABLE 9
O/W care emulsions
Quantities represent % by weight of the commercially available
substances in the composition as a whole.

| Component | 82 | 83 | 84 | 85 | 86 | 87 | 88 | 89 | 90 | 91 | 92 |
|---|---|---|---|---|---|---|---|---|---|---|---|
| L = Lotion, C = Cream | C | C | L | C | L | C | L | L | L | L | C |
| Polyalkylene ether I | 2 | | 1 | 2 | | | 1 | 1 | | | 3 |
| Polyalkylene ether III | | | | | 1 | | | | | 1 | |
| Polyalkylene ether IV | | 3 | | | | 2 | | | 1 | | |
| Eumulgin ® VL 75 | 4 | 3 | | | | | 1 | | | | 2 |
| Generol ® R | | | | | | 2 | | | | | |
| Eumulgin ® B2 | | | | | | 2 | | | | 1 | |
| Tween ® 60 | | | | | | | | | | 1 | |
| Cutina ® E 24 | | | | 2 | | | | | | | |
| Hostaphat ® KL 340 N | | | | | | | | | | | |
| Lanette ® E | 0.5 | | | | | | | | | | 1 |
| Amphisol ® K | 0.5 | 1 | | | | | | 1 | 1 | | |
| Sodium stearate | | | | | 1 | | | | | | |
| Emulgade ® PL 68/50 | | 6 | | | 1 | | | 5 | | | 4 |
| Tego ® Care CG | | | | | | | | | | | |
| Tego ® Care 450 | | | | | | | | | 4 | | |
| Cutina ® MD | 3 | | 3 | 8 | 6 | 8 | | | | 4 | |
| Lanette ® 14 | | 2 | | | | | | 2 | | 1 | |
| Lanette ® O | 2 | | | 2 | | 3 | 1 | | 1 | 1 | 6 |
| Novata ® AB | | | | | | | | | | | |
| Emery ® 1780 | | | | | | | | | | | |
| Lanolin, anhydrous, USP | | | | | | 4 | | | | | |
| Cetiol ® SB 45 | | | | | | | 2 | | | | |
| Cegesoft ® C 17 | 4 | | | | | | | | | | |
| Myritol ® PC | 6 | | | | | 5 | | | 5 | | |
| Myritol ® 331 | 5 | | 5 | | | | 7 | | | 10 | 3 |
| Finsolv ® TN | | 5 | | | 5 | | | 3 | 3 | | 1 |
| Cetiol ® CC | | | | | | | | | | | 2 |
| Cetiol ® OE | | | | | 2 | | 2 | | 5 | | |
| Dow Corning DC ® 245 | | 2 | | | 1 | | | | | 8 | 2 |
| Dow Corning DC ® 2502 | | 1 | | | 1 | | | | | | 3 |
| Prisorine ® 3758 | 3 | | | | | | | | | | 2 |
| Silikonöl Wacker AK ® 350 | | | | | 1 | | | | | | 1 |
| Cetiol ® 868 | | 2 | | | | | | | | | |
| Cetiol ® J 600 | | 2 | | | | | | | | | |
| Ceraphyl ® 45 | | | | | | | 3 | | | | |
| Cetiol ® SN | | | | | | | | | | | |
| Cetiol ® B | | | 5 | | | 5 | | 4 | | | 3 |
| Eutanol ® G | | 3 | 5 | | 5 | | | | | | |
| Cetiol ® PGL | | | | | | | | 5 | 2 | | |
| Dry Flo ® Plus | | 1 | | | | | | | | | 1 |
| SFE 839 | 1 | 1 | | | | | | | | | |
| Almond oil | | | | | | 2 | | | | | |
| Photonyl ® LS | | | | | | 2 | | | | | |
| Panthenol | 1 | | | | | | | | | | |
| Bisabolol | 0.2 | | | | | | | | | | |
| Tocopherol/Tocopheryl acetate | 1 | | | | | | | | | | |
| Veegum ® Ultra | | | | | | | | | 1 | | |
| Keltrol ® T | | | | | | | | | 0.5 | | |
| Carbopol ® ETD 2001 | | 0.3 | | 0.3 | | 0.5 | 0.2 | 0.2 | | | |
| Pemulen ® TR 2 | | | 0.3 | | | 0.3 | | | | | 0.5 |
| Ethanol | | 5 | | 8 | | | | | | | 10 |
| Butylene glycol | 5 | | 2 | 3 | 3 | | | | | 8 | |
| Glycerin | 2 | 4 | 3 | 3 | | 7 | 5 | 3 | 5 | | |

| | |
|---|---|
| Water, preservative, NaOH | to 100, q.s.,(pH 6.5-7.5) |

### APPENDIX

1) Abil® EM 90 INCI: Cetyl Dimethicone Copolyol Manufacturer: Tego Cosmetics (Goldschmidt)

2) Amphisole K INCI: Potassium Cetyl Phosphate Manufacturer: Hoffmann La Roche

3) Antarone V 220 INCI: PVP/Eicosene Copolymer Manufacturer: GAF General Aniline Firm Corp. (IPS-Global)

4) Antarone V 216 INCI: PVP/Hexadecene Copolymer Manufacturer: GAF General Aniline Firm Corp. (IPS-Global)

5) Arlacel® 83 INCI: Sorbitan Sesquioleate Manufacturer: Uniqema (ICI Surfacants)

6) Arlacel® P 135 INCI: PEG-30 Dipolyhydroxystearate Manufacturer: Uniqema (ICI Surfacants)

7) Bentone® 38 INCI: Quaternium-18 Hectorite Manufacturer: Rheox (Elementis Specialties)

8) Carbopol® 980 INCI: Carbomer Manufacturer: Goodrich

9) Carbopol® 2984 INCI: Carbomer Manufacturer: Goodrich

10)Carbopolo ETD 2001 INCI: Carbomer Manufacturer: BF Goodrich

11 )Carbopolo Ultrez 10 INCI: Carbomer Manufacturer: Goodrich

12)Cegesoft® C. 17 INCI: Myristyl Lactate Manufacturer: Cognis Deutschland GmbH, Grunau

13)Ceraphyle 45 INCI: Diethylhexyl Malate Manufacturer: International Specialty Products

14)Cetiol® 868 INCI: Ethylhexyl Stearate Manufacturer: Cognis Deutschland GmbH

15)Cetiolo A INCI: Hexyl Laurate Manufacturer: Cognis Deutschland GmbH

16)Cetiolo B INCI: Butyl Adipate Manufacturer: Cognis Deutschland GmbH (Henkel)

17)Cetiole J 600 INCI: Oleyl Erucate Manufacturer: Cognis Deutschland GmbH

18)Cetiole OE INCI: Dicaprylyl Ether Manufacturer: Cognis Deutschland GmbH

19)Cetiole PGL INCI: Hexyldecanol, Hexyldecyl Laurate Manufacturer: Cognis Deutschland GmbH

20)Cetiol CC INCI: Dicaprylyl Carbonate Manufacturer: Cognis Deutschland GmbH

21)Cetiole SB 45 INCI: Shea Butter Butyrospermum Parkii (Linne) Manufacturer: Cognis Deutschland GmbH

22)Cetiole SN INCI: Cetearyl Isononanoate Manufacturer: Cognis Deutschland GmbH (Henkel)

23)Cutinao E 24 INCI: PEG-20 Glyceryl Stearate Manufacturer: Cognis Deutschland GmbH

24)Cutina® MD INCI: Glyceryl Stearate Manufacturer: Cognis Deutschland GmbH

25)Dehymuls® FCE INCI: Dicocoyl Pentaerythrityl Distearyl Citrate Manufacturer: Cognis Deutschland GmbH

26)Dehymuls® HRE 7 INCI: PEG-7 Hydrogenated Castor Oil Manufacturer: Cognis Deutschland GmbH

27)Dehymuls® PGPH INCI: Polyglyceryl-2 Dipolyhydroxystearate Manufacturer: Cognis Deutschland GmbH

28)Dow Corning® 244 Fluid INCI: Cyclomethicone Manufacturer: Dow Corning

29)Dow Corning® 245 Fluid INCI: Cyclopentasiloxane Cyclomethicone Manufacturer: Dow Corning

30)Dow Corning® 2502 INCI: Cetyl Dimethicone Manufacturer: Dow Corning

31)Dry®Flo Plus INCI: Aluminum Starch Octenylsuccinate Manufacturer: National Starch

32)Elfacos®ST 37 INCI: PEG-22 Dodecyl Glycol Copolymer Manufacturer: Akzo-Nobel

33)Elfacos®ST 9 INCI: PEG-45 Dodecyl Glycol Copolymer Manufacturer: Akzo-Nobel

34)Emery® 1780 INCI: Lanolin Alcohol Manufacturer: Cognis Corporation (Emery)

35)Emulgade®PL 68/50 INCI: Cetearyl Glucoside, Ceteayl Alcohol Manufacturer: Cognis Deutschland GmbH

36)Eumulgin® B 2 INCI: Ceteareth- 20 Manufacturer: Cognis Deutschland GmbH

37)Eumulgin® VL 75 INCI: Lauryl Glucoside (and) Polyglyceryl-2 Dipolyhydroxystearate (and) Glycerin Manufacturer: Cognis Deutschland GmbH

38)Eusolex® OCR INCI: Octocrylene Manufacturer: Merck

39)Eusolex®T 2000 INCI: Titanium Dioxide, Alumina, Simethicone Manufacturer: Rona (Merck)

40)Eutanol®G INCI: Octyldodecanol Manufacturer: Cognis Deutschland GmbH

41)Eutanol®G 16 INCI: Hexyldecanol Manufacturer: Cognis Deutschland GmbH

42)Eutanol®G 16 S INCI: Hexyldecyl Stearate Manufacturer: Cognis Deutschland GmbH

43)Finsolv® TN INCI: C 12/15 Alkyl Benzoate Manufacturer: Findex (Nordmann/Rassmann)

44)Generol® R INCI: Brassica Campestris (Rapseed) Sterols Manufacturer: Cognis Deutschland GmbH

45)Glucate® DO INCI: Methyl Glucose Dioleate Manufacturer: NRC Nordmann/Rassmann

46)Hostaphat®KL 340 N INCI: Trilaureth -4 Phosphate Manufacturer: Clariant

47)Isolan® PDI INCI: Diisostearoyl Polyglyceryl-3 Diisostearate Manufacturer: Goldschmidt AG

48)Keltrol®T INCI: Xanthan Gum Manufacturer: CP Kelco

49)Lameform® TGI INCI: Polyglyceryl-3 Diisostearate Manufacturer: Cognis Deutschland GmbH

50)Lanette® 14 INCI: Myristyl Alcohol Manufacturer: Cognis Deutschland GmbH

51)Lanette® E INCI: Sodium Cetearyl Sulfate Manufacturer: Cognis Deutschland GmbH

52)Lanefte® O INCI: Cetearyl Alcohol Manufacturer: Cognis Deutschland GmbH

53)Monomuls® 90-0-18 INCI: Glyceryl Oleate Manufacturer: Cognis Deutschland GmbH

54)Myrj® 51 INCI: PEG-30-Sterate Manufacturer: Uniqema

55)Myritol® 331 INCI: Cocoglycerides Manufacturer: Cognis Deutschland GmbH

56)Myritol® PC INCI: Propylene Glycol Dicaprylate/Dicaprate Manufacturer: Cognis Deutschland GmbH

57)Neo Heliopan® 303 INCI: Octocrylene Manufacturer: Haarmann & Reimer

58)Neo Heliopan® AV INCI: Ethylhexyl Methoxycinnamate Manufacturer: Haarmann & Reimer

59)Neo Heliopan® BB INCI: Benzophenone-3 Manufacturer: Haarmann & Reimer

60)Neo Heliopan® E 1000 INCI: lsoamyl-p-Methoxycinnamate Manufacturer: Haarmann & Reimer

61)Neo Heliopan® Hydro (Na-Salz) INCI: Phenylbenzimidazole Sulfonic Acid Manufacturer: Haarmann & Reimer

62)Neo Heliopan® MBC INCI: 4-Methylbenzylidene Camphor Manufacturer: Haarmann & Reimer

63)Neo Heliopan® OS INCI: Ethylhexyl Salicylate Manufacturer: Haarmann & Reimer

64)Novata® AB INCI: Cocoglycerides Manufacturer: Cognis Deutschland GmbH

65)Parsol® 1789 INCI: Butyl Methoxydibenzoylmethane Manufacturer: Hoffmann-La Roche (Givaudan)

66)Pemulen® TR-2 INCI: Acrylates/C10-30 Alkylacrylate Crosspolymer Manufacturer: Goodrich

67)Photonyl® LS INCI: Arginine, Disodium Adenosine Triphosphate, Mannitol, Pyridoxine HCL, Phenylalanine, Tyrosine Manufacturer: Laboratoires Serobiologiques (Cognis)

68)Pripolo 2033 Dimer diol Manufacturer: Uniqema

69)Prisorine® ISAC 3505 INCI: Isostearic Acid Manufacturer: Uniqema

70)Prisorine® 3758 INCI: Hydrogenated Polyisobutene Manufacturer: Uniqema

71)Ravecarb® 106 Polycarbonate diol Manufacturer: Enichem

72)Ravecarb® 107 Polycarbonate diol Manufacturer: Enichem

73)SFE® 839 INCI: Cyclopentasiloxane and DimethiconeNinyl Dimethicone Crosspolymer Manufacturer: GE Silicones

74)Silikonöl Wacker AK® 350 INCI: Dimethicone Manufacturer: Wacker

75)Sovermol® 913/1 Dimerdiol-908 Manufacturer: Cognis Deutschland GmbH

76)Sovermole 920 Carbonic acid/polytetrahydrofuran polyester Manufacturer: Cognis Deutschland GmbH

77)Squatol® S INCI: Hydrogenated Polyisobutene Manufacturer: LCW (7-9 rue de l'Industrie 95310 St-Ouen I'Aumone France)

78)Tego® Care 450 INCI: Polyglyceryl-3 Methylglucose Distearate Manufacturer: Tego Cosmetics (Goldschmidt)

79)Tego® Care CG 90 INCI: Cetearyl Glucoside Manufacturer: Goldschmidt

80)Tween® 60 INCI: Polysorbate 60 Manufacturer: Uniqema (ICI Surfactants)

81)Uvinul® T 150 INCI: Octyl Triazone Manufacturer: BASF

82)Veegum® Ultra INCI: Magnesium Aluminum Silicate Manufacturer: Vanderbilt

83)Z-Cote® HP 1 INCI: Zinc Oxide, Dimethicone Manufacturer: BASF

1-16. (canceled)

17. A composition comprising a polyalkylene ether comprised of C₅₋₁₀₀ alkylene groups and having an average molecular weight of from 300 to 100000.

18. The composition according to claim 17, wherein the polyalkylene ether is prepared by polycondensation of a hydroxy-functional reactant selected from the group consisting of diols and polyols.

19. The composition according to claim 18, wherein the hydroxy-functional reactant comprises an aliphatic dihydroxy C₅₋₃₆ diol.

20. The composition according to claim 17, wherein the polyalkylene ether is prepared by polycondensation of a hydroxy-functional reactant selected from the group consisting of dimer diols, α,ω-C₅₋₃₆ alkanediols, and mixtures thereof.

21. The composition according to claim 17, wherein the polyalkylene ether is prepared by polycondensation of a hydroxy-functional reactant selected from the group consisting of α,ω-hexane diol, α,ω-dodecane diol, and mixtures thereof.

22. The composition according to claim 17, wherein the polyalkylene ether is prepared by polycondensation of a mixture of hydroxy-functional reactants comprising α,ω-hexane diol and α,ω-dodecane diol.

23. The composition according to claim 17, wherein the polyalkylene ether is prepared by polycondensation of one or more hydrogenated dimer diols having iodine values of from 20 to 80.

24. The composition according to claim 17, wherein the polyalkylene ether has an average molecular weight of from 500 to 50000.

25. The composition according to claim 17, wherein the polyalkylene ether has an average molecular weight of from 1000 to 5000.

26. The composition according to claim 19, wherein the polyalkylene ether has an average molecular weight of from 500 to 50000.

27. The composition according to claim 19, wherein the polyalkylene ether has an average molecular weight of from 1000 to 5000.

28. The composition according to claim 21, wherein the polyalkylene ether has an average molecular weight of from 500 to 50000.

29. The composition according to claim 21, wherein the polyalkylene ether has an average molecular weight of from 1000 to 5000.

30. The composition according to claim 17, further comprising at least one oil component.

31. The composition according to claim 17, further comprising at least one emulsifier.

32. The composition according to claim 31, wherein the at least one emulsifier comprises a nonionic emulsifier.

33. The composition according to claim 31, wherein the at least one emulsifier comprises an alkylpolyglycoside.

34. The composition according to claim 31, wherein the at least one emulsifier comprises an anionic emulsifier.

35. The composition according to claim 17, further comprising at least one oil component and at least one emulsifier.

36. A composition comprising: from 1 to 10% by weight of a polyalkylene ether comprised of C₅₋₁₀₀ alkylene groups and having an average molecular weight of from 300 to 100000; from 5 to 30% by weight of at least one oil component; from 0.1 to 10% by weight of at least one emulsifier; and up to 90% by weight water.
